(19) United States
(12) Patent Application Publication
(10) Pub. No.: US 2003/0054543 A1
(43) Pub. Date: Mar. 20, 2003

(54) Device for moving a selected station of a holding plate to a predetermined location for interaction with a probe

(76) Inventor: William Michael Lafferty, Encinitas, CA (US)

Correspondence Address: NEIL K. NYDEGGER, NYDEGGER & ASSOCIATES, 348 Olive Street, San Diego, CA, 92103, US

(21) Appl. No.: 10/286,194

(22) Filed: Nov. 1, 2002

Related U.S. Application Data
(63) Continuation-in-part of application No. 10/095,907, filed on Mar. 11, 2002.
Application No. 10/095,907 is a continuation-in-part of application No. 09/894,956, filed on Jun. 27, 2001.
Application No. 09/894,956 is a continuation-in-part of application No. 09/687,219, filed on Oct. 12, 2000.
Application No. 09/687,219 is a continuation-in-part of application No. 09/444,112, filed on Nov. 22, 1999.
Application No. 09/444,112 is a continuation-in-part of application No. 08/876,276, filed on Jun. 16, 1997.
(63) Continuation-in-part of application No. 09/636,778, filed on Aug. 11, 2000.
Application No. 09/636,778 is a continuation of application No. 09/098,206, filed on Jun. 16, 1998, now Pat. No. 6,174,673.
Application No. 09/098,206 is a continuation-in-part of application No. 08/876,276, filed on Jun. 16, 1997.

(51) Int. Cl. C12M 1/34; G06K 9/00

(52) U.S. Cl. 435/287.1; 382/128

(57) ABSTRACT

A device for positioning the tip of an elongated probe at a selected station of a holding plate includes motors to move the holding plate and a supporting stage within a coordinate plane (m_{xy}). The elongated probe is also moveable along a probe axis that is oriented normal to the coordinate plane (m_{xy}). A camera creates a pixel image of an optical marker placed on the stage. The image defines a coordinate plane (p_{xy}). To relate the coordinate plane (p_{xy}) to the coordinate plane (m_{xy}), the optical marker is moved to successive locations in the m_{xy} plane and a pixel image is obtained at each location. Using the pixel images, a computer calculates the relationship between coordinate planes and uses the relationship to signal the motors to move the holding plate in the m_{xy} plane and position the selected station on the probe axis for interaction with the probe.

The present application is a continuation-in-part of pending U.S. patent application Ser. No. 10/095,907 filed Mar. 11, 2002, which is a continuation-in-part of pending U.S. patent application Ser. No. 09/894,956 filed Jun. 27, 2001, which is a continuation-in-part of pending U.S. patent application Ser. No. 09/687,219, filed Oct. 12, 2000, which is a continuation-in-part of pending U.S. patent application Ser. No. 09/444,112, filed Nov. 22, 1999, which is a continuation-in-part of pending U.S. patent application Ser. No. 08/876,276, filed Jun. 16, 1997; additionally, the present application is a continuation-in-part of pending U.S. patent application Ser. No. 09/636,778, filed Aug. 11, 2000, which application is a continuation and claims the benefit of priority under 35 U.S.C. §120 of U.S. patent application Ser. No. 09/098,206, filed Jun. 16, 1998, which issued as U.S. Pat. No. 6,174,673 on Jan. 16, 2001, which is a continuation-in-part of pending U.S. patent application Ser. No. 08/876,276, filed Jun. 16, 1997, all of the contents of which are incorporated by reference in their entirety herein.

### FIELD OF THE INVENTION

The present invention pertains generally to devices for performing operations on selected samples in a holding plate with a probe. More particularly, the present invention pertains to positioning systems for moving a selected station of a holding plate to a predetermined location for interaction with a probe. The present invention is particularly, but not exclusively, useful as a computer assisted, optical system for positioning a holding plate having over a thousand, small diameter through-hole stations at a precise location to allow a probe to interact with a selected station.

### BACKGROUND OF THE INVENTION

Plates for holding specimen samples in a fluid solution are available having over a thousand, small diameter stations. The stations can include through-holes, or wells that extend only partially into the holding plate. In the case of a through-hole station, these stations rely on surface tension to hold each fluid sample in a respective station. The through-hole stations of a holding plate can be filled with a solution of interest by simply immersing a surface of the holding plate into the solution. Capillary action causes the solution to enter the through-hole stations. This allows a very large number of relatively small volume samples of a solution to be simultaneously prepared for later analysis or manipulation. Specifically, holding plates having over a thousand stations arranged in a planar array, with station diameters of only about 500 microns, are available.

Once the holding plate has been filled with solution, it is often desirable to either add material to selected stations or withdraw solution from selected stations. This is particularly the case when the solution used to fill the holding plate is non-homogenous. Often times, the selected stations differ in color, opacity, fluorescence or are otherwise optically distinguishable from the remaining stations. For example, a biological or chemical reaction may proceed more rapidly in portions of the solution, causing only selected stations to change color, while the remaining stations do not. Withdrawal of solution from the selected stations allows for the separation of the solution into portions of solution that have reacted and portions of solution that have not reacted. Alternatively, it may be desirable to add a material such as a chemical reagent to selected stations, again selecting stations based on some optical property of the sample in the station. In still another application, it may be desirable to initiate a different chemical or biological reaction in each station resulting in one or more stations that are optically distinguishable from the other stations.

Generally, a thin, needle-like probe must be positioned in fluid communication with a selected station to either add or withdraw material from the station. Thus, it is often desirable to select a specific station and then operate on the selected station with a probe. To accomplish this, the probe and selected station must first be aligned. Unfortunately, for stations having extremely small diameters, such as through-holes with diameters of 500 microns or less, it is impossible for all practical purposes, to align a selected station with a probe using the naked eye. Furthermore, for holding plates having a thousand or more stations, systems that require human interaction to align the probe with each station are too slow to be practical. Thus, the present invention recognizes that a computer-assisted, automated system is necessary to align small diameter stations with a probe.

Probe interaction with stations having diameters of only about 500 microns requires the location of the probe to be known with great certainty. Small changes in the position of the probe must be taken into consideration in order to properly align the probe with the small stations. For example, each time a probe is replaced or serviced can result in a small change in probe location that must be considered. A convenient and accurate way to determine the position of the probe is to image the probe. When sample imaging and probe interaction with the sample are performed on different surfaces of the holding plate, use of a single imaging system is straightforward. For example, co-pending U.S. application Ser. No. 10/095,907 for an invention entitled “A Positioning System for Moving a Selected Station of a Holding Plate to a Predetermined Location for Interaction with a Probe” discloses and claims such a system. However, when sample imaging and probe interaction with the sample are performed on the same surface of the holding plate, a more complicated system is required.

Holding plates are generally designed with stations (i.e. through-holes or wells) having station axes that are perpendicular to the surfaces of the holding plate. With this design, the axes of the stations are relatively easily aligned with the path of the probe. Unfortunately, due to defects in the manufacturing processes that are used to prepare the holding plates, the axes of the stations can sometimes be misaligned, albeit slightly, from the surfaces of the holding plate. Stated another way, an end of the station on one surface of a holding plate is offset from the other end of the station on the opposite surface of the holding plate. It is to be appreciated that this offset can present problems when imaging is performed on one surface of the holding plate while the probe is aligned with the station on the opposite surface of the holding plate. The problem becomes more egregious with respective increases in the aspect ratio of the station, the density of stations on the plate and the thickness of the plate. One way to overcome the problems associated with misaligned stations is to perform imaging and probe alignment on the same surface of the holding plate.

It is often the case that hundreds of stations (among the thousand or more stations present in the holding plate) may require interaction with the probe. In these cases, it becomes too labor intensive for an operator to select each station individually for interaction with the probe. Thus, it would be desirable to have a computer-assisted system that allows the operator to select a set of stations by merely choosing an optical characteristic to establish the set. With the set established, the operator then instructs the computer to successively perform a probe operation on each station in the selected set. A convenient system would allow an operator to specify an optical characteristic; for example, fluorescence, and then instruct the computer to make a chemical addition to each station having a sample that is fluorescing.

In light of the above, it is an object of the present invention to provide a system suitable for the purposes of moving a selected station of a holding plate to a predetermined location for interaction with a probe. It is another object of the present invention to provide a positioning system for aligning a probe and selected station wherein the station has an extremely small diameter (i.e. a through-hole having a diameter of 500 microns or less). It is yet another object of the present invention to provide a system for automatically performing a probe operation on samples in a selected set of stations that all have a common optical characteristic. Still another object of the present invention is to provide a positioning system for aligning a probe with a selected station wherein the station axis is offset (i.e. at a non-normal angle) relative to the surfaces of the holding plate. Another object of the present invention is to provide a positioning system for aligning a probe with a selected station wherein holding plate imaging and probe alignment are performed on the same surface of the holding plate. Yet another object of the present invention is to provide an optical positioning system for aligning a small diameter through-hole station with a probe which can be used on opaque plates, is relatively simple to implement, and comparatively cost effective.

### SUMMARY OF THE INVENTION

The present invention is directed to devices for positioning a holding plate to allow the tip of a probe to interact with a selected station of the plate. For the present invention, the holding plate is formed with a substantially planar first surface and an opposed second surface. Preferably, the holding plate is further formed with a regular or irregular planar array of stations for holding a plurality of respective samples. Importantly, each station is accessible by the probe from the first surface of the holding plate.

In accordance with the present invention, the probe is attached to a base and a mechanism is provided to allow for reciprocal movement of the probe relative to the base. The device further includes a moveable stage that is mounted on the base to support the holding plate. For the present invention, the moveable stage is formed with a planar surface for engagement with the second surface of the holding plate. With this cooperation of structure, the planar surface of the stage defines a coordinate plane (m_{xy}) containing orthogonal axes x and y. A mechanism is provided to secure the holding plate to the stage, causing the holding plate to move with the stage. With the second surface of the holding plate secured against the stage, the first surface of the holding plate remains exposed for interaction with the probe. To selectively move the stage (and the holding plate) in the x and y directions relative to the base and probe, the device further includes a pair of motorized linear actuators.

As indicated above, the probe is attached to the base. In greater structural detail, the probe includes an elongated portion that defines a probe axis in the direction of elongation. In one embodiment, the elongated probe is optically distinguishable and, for this purpose, is mounted on a fluorescent hub and extends from the fluorescent hub to a probe tip. The hub, in turn, is mounted on the base. Importantly for the present invention, the probe is positioned relative to the holding plate to allow the tip of the probe to interact with the first surface of the holding plate. Additionally, the probe and hub are preferably mounted on the base with the probe axis of the probe oriented normal to the m_{xy} plane. In the preferred embodiment of the present invention, a mechanism is provided to allow the probe to reciprocate (relative to the holding plate and base) along the probe axis and in a direction that is substantially orthogonal to the m_{xy} plane. Once installed, the probe does not move in the m_{xy} plane. With the above-described combination of structure, the motorized linear actuators can be used to move the holding plate to a location in the m_{xy} plane such that a selected station is positioned on the probe axis. With the selected station positioned on the probe axis, the probe can be moved along the probe axis to interact with the selected station.

To locate a selected station of the holding plate at a position on the probe axis, the device includes at least one camera and a computer processor. In one embodiment of the present invention, the camera is positioned on the probe axis and oriented to obtain a pixel image of the holding plate stations from the second surface of the holding plate. To facilitate imaging from the second surface of the holding plate, a transparent stage is preferably used. Alternatively, one or more holes can be formed in the stage to allow the camera to image the stations from the second surface of the holding plate.

In another embodiment, the camera is positioned to obtain a pixel image of the first surface of the holding plate. This cooperation of structure (i.e. imaging and manipulation on the same surface of the holding plate) eliminates positioning errors due to offset stations. In this embodiment, a single camera can be used to image both the holding plate and the probe tip. During imaging of the first surface of the holding plate, the probe is distanced from the first surface of the holding plate and thus does not interfere with imaging. To image and determine the location of the probe tip, a mirror is mounted on the stage to establish an optical path between the probe tip and the camera. When the location of the probe is required, the stage and mirror are moved to a pre-selected position where the probe tip will appear in the image generated by the camera. The image can then be processed to determine the location of the probe tip relative to the base.

In operation, the device is initially calibrated (calibration procedure described below). Next, a first holding plate is installed on the stage, placing the holding plate at a first location in the m_{xy} plane. One or more pixel images are then obtained by the camera that images the array of stations positioned at the first location in the m_{xy} plane. The projection of the probe in the m_{xy} plane is either obtained directly when the holding plate is imaged on the second surface of the holding plate or via the stage mounted mirror when the plate is imaged on the first surface of the holding plate.

For the present invention, the pixel image defines a coordinate plane (p_{xy}) that is related to the coordinate plane (m_{xy}). From the pixel image, the operator selects a specific station of the holding plate that requires interaction with the probe. This information is then transferred to the computer processor. The computer processor instructs the motorized linear actuators to move the holding plate through the proper x and y distances in the m_{xy} plane to align the selected station on the probe axis. More specifically, the computer uses a relationship that was previously established between the coordinate plane (p_{xy}) and the coordinate plane (m_{xy}) during calibration to accurately move the stage and align the selected station on the probe axis. With the selected station positioned on the probe axis, the probe is then translated along the probe axis to interact with the station. When the holding plate is imaged from the second surface of the holding plate, station offset information (i.e. the deviation of each station axis from a reference axis that is orthogonal to the surface of the holding plate) can be inputted into the computer processor. The computer processor can then use the offset information to ensure that the station entrance located at the first surface of the holding plate is aligned with the probe axis.

To calibrate the device, an optical marker is placed on the stage and a first pixel image is obtained by the camera. As such, the first pixel image includes the optical marker positioned at a first location in the m_{xy} plane. Next, the stage is moved using the motorized linear actuators to successive locations in the m_{xy} plane. The actuator displacements (e.g. motor steps) necessary to move the optical marker between locations are recorded and a pixel image of the optical marker is obtained at each location. These pixel images and actuator displacements are then used by the computer processor to correspond the p_{xy} coordinate plane with the m_{xy} coordinate plane. Stated another way, the pixel images are used to find the relationship between the p_{xy} coordinate plane and the m_{xy} coordinate plane. Preferably, the method of least squares is used to establish an approximate linear relationship between the coordinate plane (p_{xy}) and the coordinate plane (m_{xy}).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a device for moving a selected station of a holding plate to a predetermined location for interaction with a probe;

FIG. 2 is an enlarged, sectional view of a portion of a holding plate and stage as would be seen along line 2-2 in FIG. 1;

FIG. 3A is an exemplary pixel image taken after the optical marker has been moved to a first location;

FIG. 3B is an exemplary pixel image taken after the optical marker has been moved to a second location;

FIG. 3C is an exemplary pixel image taken after the optical marker has been moved to a third location;

FIG. 4 is a sectional view as in FIG. 2 showing a holding plate with offset stations;

FIG. 5 is a perspective view of another embodiment for moving a selected station of a holding plate to a predetermined location for interaction with a probe in which one surface of the holding plate is used for both imaging and interaction with the probe;

FIG. 6 is a front elevation view of the device shown in FIG. 5 showing the stage positioned to allow imaging of the holding plate; and

FIG. 7 is a front elevation view as in FIG. 6 shown after the stage has been moved to position the mirror along the camera's beam path to allow the location of the probe tip to be determined.

### DESCRIPTION OF THE PREFERRED EMBODIMENT

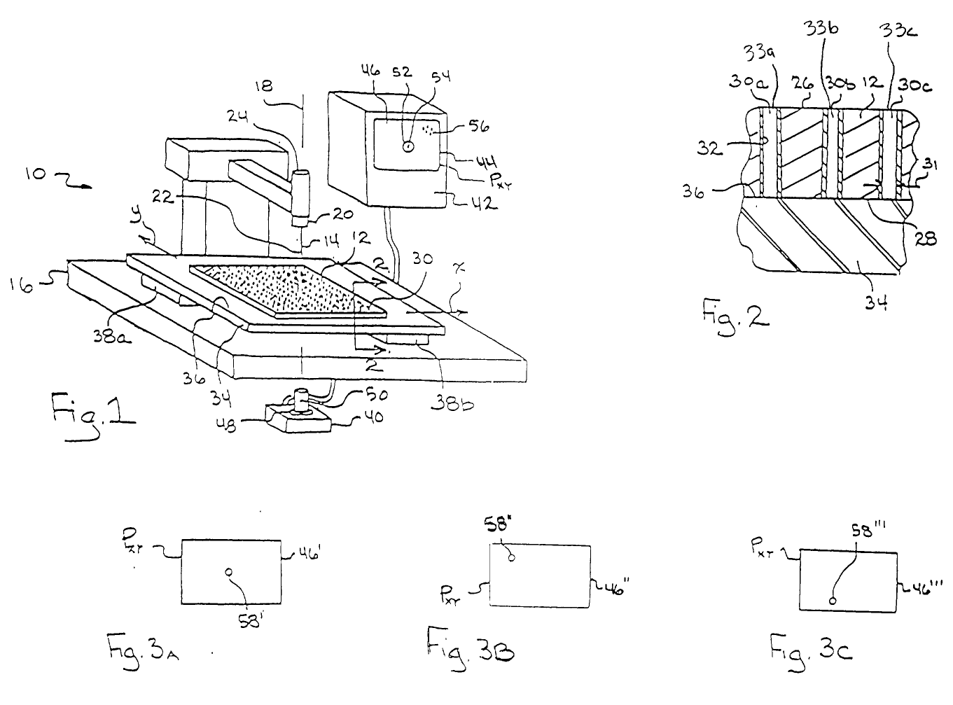

Referring initially to FIG. 1, a device 10 for performing operations on selected samples in a holding plate 12 with a probe 14 is shown. As shown, the device 10 includes a base 16 for supporting both the holding plate 12 and the probe 14. As further shown, the probe 14 is elongated and defines a probe axis 18 in the direction of elongation. Typically, the probe 14 is formed as a hollow needle having a lumen capable of transferring fluid. Also shown in FIG. 1, the elongated probe 14 is mounted on a hub 20 and extends from the hub 20 to a probe tip 22. The hub 20, which is preferably fluorescent, is somehow optically distinguishable to differentiate the probe 14 from the hub 20. The device 10 also includes a mechanism 24 to move the probe 14 back and forth along the probe axis 18, relative to the base 16 and holding plate 12. Those skilled in the art will appreciate that the mechanism 24 for reciprocating a probe back and forth along an axis could be a hydraulic or pneumatic cylinder or any other similar mechanism known in the pertinent art.

With cross reference now to FIGS. 1 and 2, it can be seen that the holding plate 12 is formed with a substantially planar first surface 26 and an opposed second surface 28. The holding plate 12 can be formed with a regular or irregular planar array of stations 30, for which stations 30a-c shown in FIG. 2 are exemplary. Each station 30 is provided to hold a fluid sample and may be a through-hole that extends through the plate 12 between surfaces 26 and 28 as shown, or a well having a bottom within the plate (not shown). A typical holding plate 12 can be formed with over one thousand stations 30, with each station 30 having an inner diameter 31 of approximately 500 microns or less. An optional coating 32 can be applied to each through-hole station 30 to limit the transmission of light between adjacent stations 30. As further shown in FIG. 2, each station 30a-c has a respective station entrance 33a-c allowing the each station 30 to be accessed by the probe 14 from the first surface 26 of the holding plate 12.

With continued cross reference to FIGS. 1 and 2, it can be seen that the device 10 further includes a moveable stage 34 that is mounted on the base 16 to support the holding plate 12. As further shown, the moveable stage 34 is formed with a planar surface 36 for engagement with the second surface 28 of the holding plate 12. As shown, the planar surface 36 of the stage 34 defines a coordinate plane (m_{xy}) containing orthogonal axes x and y. If required, clamps (not shown) can be provided to secure the holding plate 12 to the stage 34. In any case, with the holding plate 12 on the stage 34, the stage 34 and holding plate 12 move together. With the second surface 28 of the holding plate 12 secured against the stage 34, the first surface 26 of the holding plate 12 remains exposed for interaction with the probe 14.

As best seen in FIG. 1, the device 10 includes a pair of motorized linear actuators 38a, b that are mounted on the base 16 to selectively move the stage 34 and holding plate 12 in the x and y directions relative to the base 16 and probe 14. It is to be further appreciated that the motorized linear actuators 38a, b move the holding plate 12 within the m_{xy} plane. A suitable motorized linear actuator 38a, b includes a stepper motor for driving a lead screw to move the stage 34. However, those skilled in the pertinent art will appreciate that any type or number of motorized linear actuators or other devices known in the pertinent art for selectively moving a stage in at least two directions can be used.

Referring now with cross reference to FIGS. 1 and 2, it can be seen that the probe 14 is positioned relative to the holding plate 12 to allow the probe tip 22 to interact with the first surface 26 of the holding plate 12. Additionally, the probe 14 is mounted on the base 16 with the probe axis 18 of the probe 14 oriented normal to the m_{xy} plane (i.e. the plane containing the x and y axes). Thus, the probe 14 reciprocates along the probe axis 18 and in a direction that is orthogonal to the m_{xy} plane. The motorized linear actuators 38a, b can be selectively activated to move the holding plate 12 to a location in the m_{xy} plane such that a selected station 30 is positioned on the probe axis 18. With the selected station 30 positioned on the probe axis 18, the probe 14 can then be moved along the probe axis 18 to interact with the selected station 30. More specifically, the probe 14 can manipulate a sample that is held by the holding plate 12 at the selected station 30. Manipulations of the sample by the probe 14 can include sample withdrawal from the station 30 or the addition of a material such as a chemical reagent to the sample.

As best seen in FIG. 1, the device 10 includes a camera 40 and a computer processor 42 with a display 44. In the embodiment shown in FIG. 1, the camera 40 is positioned on the probe axis 18 and oriented to image the stations 30 of the holding plate 12 from the second surface 28 (shown in FIG. 2) of the holding plate 12. The camera 40 produces a pixel image 46 that can be displayed on the display 44. The holding plate 12 can be imaged through transparent portions of the stage 34 and base 16, or one or more holes can be formed in the stage 34 and base 16.

Referring still to FIG. 1, the device 10 can include an illumination system 48 for illuminating and/or exciting samples in the holding plate 12. For example, the illumination system 48 can be used to excite fluorescent materials in the holding plate 12. In accordance with the present invention, one or more light filters 50 can be used to selectively filter light entering the camera 40. For example, light filter 50 can be used to filter out backscattered excitation light from illumination system 48 while allowing fluorescent emissions from the samples to be imaged by the camera 40.

In operation, a holding plate 12 is installed on the stage 34, as shown in FIG. 1 and a pixel image 46 is created by camera 40 and presented in a viewable format by display 44. As shown, the pixel image 46 sequentially includes a hub image 52, a probe image 54 and an image of the array of stations 30 of the holding plate 12. In part, because the probe 14 is surrounded by an optically distinguishable hub 20, the probe tip 22 of the relatively thin probe 14 can be accurately imaged. It is to be appreciated that the pixel image 46 also shows stations 30, including stations 30 that have distinguishing optical characteristics (e.g. color, fluorescence, opacity, etc). In FIG. 1, pixel image 46 shows the image of five selected stations 30 that have distinguishing optical characteristics (i.e. selected stations image 56).

As indicated above, the function of the device 10 is to move the holding plate 12 within the m_{xy} plane to position a selected station 30 on the probe axis 18. With the selected station 30 on the probe axis 18, the probe 14 is then moved along the probe axis 18 to manipulate a sample in the selected station 30. The pixel image 46 defines a coordinate plane (p_{xy}) that is related to the coordinate plane (m_{xy}). In one implementation of the device 10, stations 30 are selected in the pixel image 46 for manipulation by the probe 14. The computer processor 42 then instructs the motorized linear actuators 38a, b to move the holding plate 12 within the m_{xy} plane to position the selected station 30 on the probe axis 18. The device 10 is calibrated to accomplish this movement with extremely small positional errors. During calibration, the computer processor 42 determines the relationship (i.e. correspondence) between the coordinate plane (p_{xy}) and the coordinate plane (m_{xy}).

To establish the relationship between the coordinate plane (p_{xy}) and the coordinate plane (m_{xy}), an optical marker is placed on the stage 34 and the stage 34 is moved via the motorized linear actuators 38a, b to successive locations in the m_{xy} plane. A separate pixel image 46 is obtained at each location. The displacements of the motorized linear actuators 38a, b (e.g. motor steps) necessary to move the optical marker from the first location to the second location and from the second location to the third location are recorded and input into the processor 42.

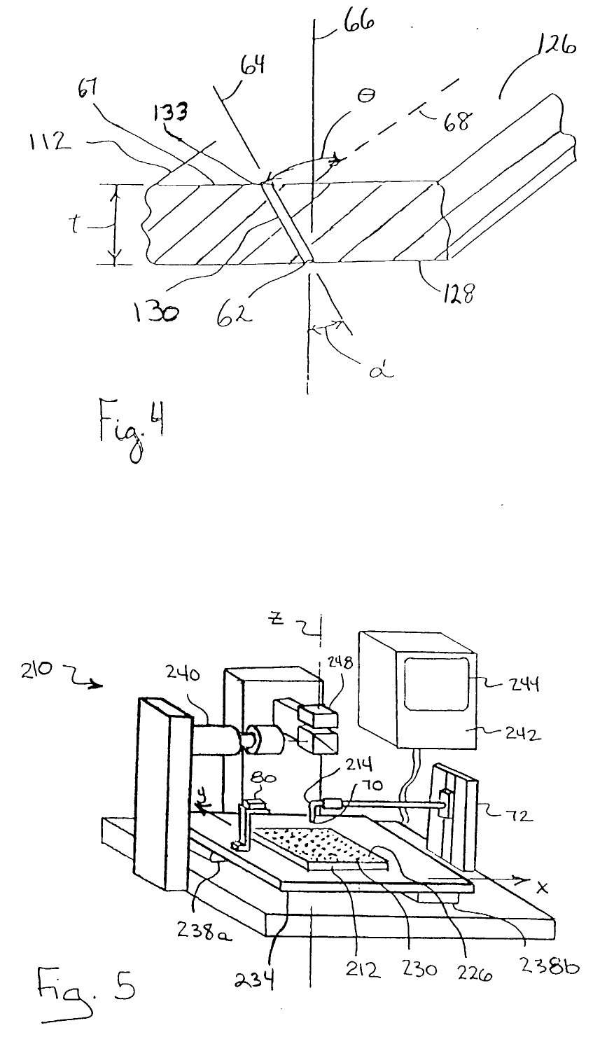

FIGS. 3A, 3B and 3C show pixel images 46′, 46″ and 46′″ for three locations of the stage 34 within the m_{xy} plane. In greater detail, FIG. 3A shows pixel image 46′ for stage 34 in a first location and includes an optical marker image 58′. Similarly, FIG. 3B shows pixel image 46″ for stage 34 in a second location and includes an optical marker image 58″. Also, FIG. 3C shows pixel image 46′″ for stage 34 in a third location and includes an optical marker image 58′″. Although pixel images 46′, 46″ and 46′″ for three stage 34 locations are shown herein, it is to be appreciated that any number of locations can be used with the present invention to establish a relationship between the coordinate plane (p_{xy}) and the coordinate plane (m_{xy}). Once the displacements of the motorized linear actuators 38a, b (e.g. motor steps) and pixel images 46′, 46″ and 46′″ have been obtained, a linear regression technique, such as the method of least squares, can be used by the processor 42 to establish an approximate linear relationship between the coordinate plane (p_{xy}) and the coordinate plane (m_{xy}) to calibrate the device 10.

Referring now to FIG. 4, a portion of a holding plate 112 having a thickness, “t”, is shown. The holding plate 112 includes a station 130 with a station entrance (top) 133 that is offset from the station exit (bottom) 62. As further shown, the characteristic axis 64 of the station 130 is inclined at an angle, α, from an axis 66. More specifically, the axis 66 is normal to the first surface 126 of the holding plate 112 and passes through the exit (bottom) 62. It can be further seen that a line 67 on first surface 126, which intersects both the axis 66 and the axis 64 establishes a rotation angle, θ, between the line 67 and a base reference line 68 about the axis 66. When the second surface 128 of the holding plate 112 is imaged and the first surface 126 is used for access by the probe 14 (as shown in FIG. 1), this offset information (i.e. α, θ, and “t”) for the plate 112 is input into the computer processor 42. With this offset information, the computer processor 42 uses an image of the second surface 128 of the plate 112 to accurately locate the entrance 60 of the plate 112 on the probe axis 18 (probe axis 18 shown in FIG. 1).

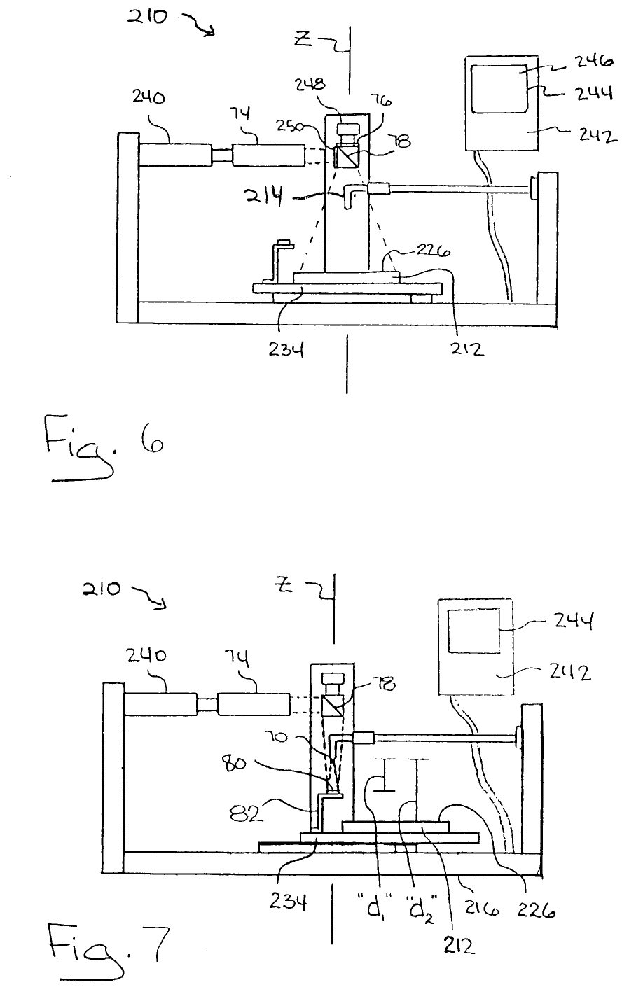

FIG. 5 shows another embodiment of a device (hereinafter device 210) for performing operations on selected samples in a holding plate 212 with a probe 214. In this embodiment, a pixel image of the first surface 226 of the holding plate 212 is obtained by camera 240 and samples in stations 230 are manipulated by the probe 214 from the first surface 226. This cooperation of structure (i.e. imaging and manipulation from the same surface of the holding plate 212) eliminates positioning errors due to offset stations 230. In greater detail, it can be seen from FIG. 5 that the probe 214 includes a ninety degree bend and extends from the bend substantially along a z-axis to a probe tip 70. As further shown, a mechanism 72 is provided to reciprocate the probe tip 70 back and forth along the z-axis.

With cross-reference to FIGS. 5 and 6, it can be seen that the first surface 226 of the holding plate 212, including stations 230 and optical markers for calibration, can be imaged using camera 240 having lens 74. The resultant pixel image 246 can be presented on display 244. The device 210 can include an illumination system 248 for illuminating and/or exciting samples in the holding plate 212. For example, the illumination system 248 can be used to excite fluorescent materials in the holding plate 212. As shown, light from the illumination system 248 can be filtered using filter 76 and then directed through half-silvered mirror 78 to the holding plate 212.

Continuing with FIGS. 5 and 6, it can be seen that light from holding plate 212 reflects from half silvered mirror 78 and enters camera 240 via lens 74. As shown, one or more light filters 250 can be positioned to selectively filter light entering the camera 240. For example, light filter 250 can be used to filter out backscattered excitation light from illumination system 248 while allowing fluorescent emissions from the samples to be imaged by the camera 240. It can be further seen from FIG. 6 that during imaging of the first surface 226 of the holding plate 212, the probe 214 is sufficiently removed from the object plane (i.e. the plane of first surface 226) and thus does not interfere with imaging of the first surface 226 of the holding plate 212.

Referring now to FIG. 7, the device 210 is shown configured to image, and determine the location of, the probe tip 70. For this purpose, the device 210 includes a mirror 80 that is mounted on the stage 234 via bracket 82. Movement of the stage 234 allows the mirror 80 to be positioned along the z-axis as shown in FIG. 7. In this position, the probe tip 70 can be imaged and the image used by the processor 242 to determine the location of the probe tip 70 and z-axis relative to the base 216. In greater detail, as shown, light rays from the probe tip 70 are reflected by the mirror 80 into the half silvered mirror 78 where the rays are reflected into the camera 240 via lens 74. In one implementation of the device 210, the location of the probe tip 70 is determined each time the probe tip 70 is replaced and at periodic intervals between replacements. In a particular embodiment of the device 210, the mirror 80 is positioned at a distance “d₁” from the probe tip 70 that is approximately one-half the distance “d₂” between the probe tip 70 and the first surface 226 of the holding plate 212. With this cooperation of structure, the effective optical length between the lens 74 and probe tip 70 is substantially equal to the effective optical length between the lens 74 and first surface 226 of the holding plate 212.

Once the position of the probe tip 70 (and z-axis) is determined, a holding plate 212 is installed on the stage 234, as shown in FIG. 5. The device 210 is then calibrated by imaging the position of an optical marker on the holding plate 212 at several locations to determine the relationship (i.e. correspondence) between the coordinate plane (p_{xy}) and the coordinate plane (m_{xy}) as described above. After calibration of the device 210, the device 210 can then be used to move the holding plate 212 within the m_{xy} plane to position a selected station 230 on the z-axis. Specifically, the computer processor 242 instructs the motorized linear actuators 238a, b to move the holding plate 212 within the m_{xy} plane to position the selected station 230 on the z-axis. With the selected station 230 on the z axis, the probe tip 70 is then moved along the z-axis to manipulate a sample in the selected station 230.

While the particular device for moving a selected station of a holding plate to a predetermined location for interaction with a probe as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for manipulating samples at respective stations of a holding plate, said stations having a station entrance on a first planar surface of said holding plate, said device comprising:
a base;
a probe mounted on said base, said probe having a probe tip for reciprocative movement along a z-axis to interact with said station entrances on said first planar surface of said holding plate;
a stage mounted on said base for supporting said holding plate;
a motor for moving said stage in a first coordinate plane (m_{xy}) orthogonal to said z-axis;
a detection means for locating said z-axis and said station entrances on said first planar surface of said holding plate in a second coordinate plane (p_{xy}); and
a computer means for corresponding said first coordinate plane with said second coordinate plane, said computer means being coupled with said motor to align said stage with said probe for movement of said probe to a selected station entrance of said holding plate for manipulating a sample at said selected station.

2. A device as recited in claim 1 wherein said detection means comprises a camera for creating a pixel image and wherein said camera is positioned to receive light that is directed away from said holding plate from said first surface of said holding plate.

3. A device as recited in claim 2 wherein said detection means comprises a mirror mounted on said stage for focusing said camera on said probe tip to locate said z-axis in said second coordinate plane (p_{xy}).

4. A device as recited in claim 3 further comprising an illumination system for causing a portion of said samples to fluoresce for detection and viewing thereof by said camera.

5. A device as recited in claim 4 further comprising an optical filter to prevent backscattered light from said illumination system from reaching said camera.

6. A device as recited in claim 5 wherein said probe is formed with a bend of approximately ninety degrees (90°).

7. A device as recited in claim 1 wherein said computer means corresponds said first coordinate plane with said second coordinate plane using least squares techniques.

8. A device as recited in claim 1 wherein said holding plate has more than one thousand said stations.

9. A device for manipulating samples at respective stations of a holding plate, said device comprising:
a motorized means for moving said holding plate in a first coordinate plane (m_{xy});
a probe having a probe tip;
means for reciprocating said probe tip along a z-axis orthogonal to said first coordinate plane (m_{xy}) to interact with a first surface of said holding plate;
a detection means for imaging said first surface of said holding plate and said z-axis in a second coordinate plane (p_{xy}); and
a computer means for corresponding said first coordinate plane with said second coordinate plane to control the movement of said holding plate by said motorized moving means to position a selected said sample along said z-axis for manipulation of said selected sample by said probe.

10. A device as recited in claim 9 wherein said detection means comprises a camera for creating a pixel image.

11. A device as recited in claim 10 wherein said detection means further comprises a mirror mounted on said stage for focusing said camera on said probe tip to locate said z-axis in said second coordinate plane (p_{xy}).

12. A device as recited in claim 11 further comprising an illumination system for causing a portion of said samples to fluoresce for detection and viewing thereof by said camera.

13. A device as recited in claim 12 further comprising an optical filter to prevent backscattered light from said illumination system from reaching said camera.

14. A device as recited in claim 9 wherein said computer means corresponds said first coordinate plane with said second coordinate plane using least squares techniques.

15. A method for manipulating a sample at a selected station of a holding plate, said method comprising the steps of:
providing a probe having a probe tip;
positioning said holding plate for movement in a first coordinate plane (m_{xy});
imaging a first surface of said holding plate in a second coordinate plane (p_{xy});
establishing a relationship between said first coordinate plane (m_{xy}) and said second coordinate plane (p_{xy});
using said relationship to move said holding plate in said first coordinate plane (m_{xy}) to position said selected sample at a predetermined location in said first coordinate plane;
reciprocating said probe tip along an axis orthogonal to said first coordinate plane (m_{xy}) to interact with said selected sample from said first surface of said holding plate; and
manipulating said sample using said probe.

16. A method as recited in claim 15 further comprising the step of creating an image having an image of said probe tip to determine the location of said probe tip.

17. A method as recited in claim 16 wherein said holding plate is attached to a moveable stage and said step of creating an image having an image of said probe tip includes the step of moving said stage to position a mirror to create an optical path between said probe tip and a camera.

18. A method as recited in claim 15 wherein said step of establishing a relationship between said first coordinate plane (m_{xy}) and said second coordinate plane (p_{xy}) comprises the following steps:
attaching an optical marker to said holding plate;
imaging said holding plate with said optical marker at a first location in said first coordinate plane (m_{xy}) to obtain a first image location for said optical marker;
moving said holding plate and said optical marker to a second location in said first coordinate plane (m_{xy});
measuring the distances along a set of orthogonal axes between said first location and said second location in said first coordinate plane (m_{xy});
imaging said holding plate with said optical marker at said second location in said first coordinate plane (m_{xy}) to obtain a second image location for said optical marker;
calculating the distances along a set of orthogonal axes between said first image location and said second image location in said second coordinate plane (p_{xy}); and
comparing said measured distances to said calculated distances to determine the relationship between said first coordinate plane (m_{xy}) and said second coordinate plane (p_{xy}).

19. A method as recited in claim 15 wherein said step of manipulating said sample using said probe comprises adding a material to said sample.

20. A method as recited in claim 15 wherein said step of manipulating said sample using said probe comprises withdrawing material from said sample.
